(19) United States
(12) Patent Application Publication
(10) Pub. No.: US 2020/0188240 A1
(43) Pub. Date: Jun. 18, 2020

(54) Personalized Topical Application Patch

(71) Applicant: Johnson & Johnson Consumer Inc., Skillman, NJ (US)

(72) Inventors: Jonathan Hansen, Jacksonville, FL (US); Peyton Hopson, Jacksonville, FL (US); Dianne Rossetti, Skillman, NJ (US); Michael Southall, Skillman, NJ (US)

(21) Appl. No.: 16/718,961

(22) Filed: Dec. 18, 2019

Related U.S. Application Data
(60) Provisional application No. 62/781,115, filed on Dec. 18, 2018.
(60) Provisional application No. 62/861,109, filed on Jun. 13, 2019.

(51) Int. Cl. A61K 8/02; A61K 9/70; A61K 47/42; A61Q 19/00; A61K 8/67; A61B 5/00; G16H 30/20

(52) U.S. Cl. CPC A61K 8/0212; A61K 9/703; A61K 47/42; G16H 30/20; A61K 8/675; A61B 5/0064; A61Q 19/00

(57) ABSTRACT

A personalized topical application patch includes a patch substrate having a plurality of isolated regions; and one or more active benefit agents disposed at least one of the plurality of isolated regions.

### FIELD OF THE INVENTION

The present invention relates to devices and methods for producing and providing to a user a personalized topical application patch with spatially isolated regions for active benefit agents.

### BACKGROUND

Currently, many actives are applied to substrates in bulk whereby there is no location specific applications. This limits the customization within and on the surface of a substrate. As such, specific treatment is administered across the entire bulk or surface of the article. The problems inherent in the foregoing include (1) excessive use of actives (i.e., application is applied to unnecessary areas); (2) potential adverse events from active in unintended areas; (3) reduced loading at targeted application site; (4) negative interactions between benefit agents or incompatible benefit agents and (5) limitations in product personalization.

Therefore, what is needed is customization of the spatial arrangement of actives on a topical application patch, especially in materials often used for active delivery in which high concentrations of the active and rapid diffusion are required (i.e., hydrogels) in a manner to prevent migration of the actives from their predetermined location on the substrate.

### SUMMARY OF THE INVENTION

It has been discovered that the manufacture and supply of personalized topical application patches can be addressed in a surprising and different way with a uniquely segregated spatial arrangement of active benefit agents disposed thereon.

In one embodiment of the invention, a method for providing a personalized topical application patch to a person includes the steps of:
a) acquiring body-surface data of a person;
b) communicating the body-surface data to a design creation system which produces a digital design file corresponding to the body-surface data;
c) communicating the digital design file to a manufacturing site;
d) forming a personalized topical application patch by:
   i) placing a patch substrate on a carrier;
   ii) forming at least one barrier in the patch substrate to define at least two discrete regions of the patch substrate;
   iii) applying one or more active benefit agents onto at least one of the discrete regions of the patch substrate; and
   iv) cutting the patch substrate to a desired shape for the person and removing waste material patch substrate material; wherein the barriers are substantially impervious to diffusion of the one or more active benefit agents;
e) packaging the personalized topical application patch; and
f) delivering the personalized topical application patch to the person.

In another embodiment of the invention, a personalized topical application patch includes a patch substrate having a plurality of isolated regions; and one or more active benefit agents disposed at least one of the plurality of isolated regions. There may be at least one barrier disposed between adjacent isolated regions, where the barrier is substantially impervious to the diffusion of the one or more active benefit agents.

### BRIEF DESCRIPTION OF THE DRAWING

FIG. 1 shows a schematic flow diagram of a system to provide personalized topical application masks to a consumer according to the present invention.

FIG. 2 shows a plan view of a personalized topical application patch in the form of a facial mask, according to the present invention.

FIG. 3 shows three steps of a method of forming a personalized topical application patch according to one embodiment of the present invention; each step shows a cross-section of a representative portion of the personalized topical application patch or one or more components thereof.

FIG. 4 shows three steps of a method of forming a personalized topical application patch according to one embodiment of the present invention; each step shows a cross-section of a representative portion of the personalized topical application patch or one or more components thereof.

FIG. 5 shows cross-section of a portion of a personalized topical application patch according to one embodiment of the present invention.

FIG. 6 shows three steps of a method of forming a personalized topical application patch according to one embodiment of the present invention; each step shows a cross-section of a representative portion of the personalized topical application patch or one or more components thereof.

### DETAILED DESCRIPTION OF THE INVENTION

As used herein the specification and the claims, the term “topical” and variants thereof mean of or applied to an isolated part of the body. This includes, without limitation skin, mucosa, hair, nails, and enamel.

We have developed a system to deliver a personalized topical application patch to an individual user. In particular, a user can scan a region of body surface, such as a face, to obtain body-surface data including the identification of regions of the body surface and associated skin-improvement opportunities for such regions. Scans, including 3D scans, of a body surface, such as a face, can be obtained by using an infrared emitter in a device such as a smartphone. By projected thousands of dots in a known pattern across a subject's face, these dots can be captured with digital photography using a camera with an infrared sensor and analyzed. Measuring skin conditions which are in a depth dimension, such as wrinkles/fine lines, skin texture/roughness, and acne lesions, can be difficult or inaccurate when using a 2D scan from standard photography or imaging. Also, additional objects on the skin, such as stray hairs, could be interpreted as fine lines in 2D imaging, giving a false positive response and cause a system to attempt to address a non-existent skin defect, because the 2D image cannot differentiate a hair from a wrinkle as well as a 3D image can.

A 3D image of the body region, or face, can then be rendered to accurately capture distance between points such as the eyes, and forehead to chin. The 3D image of the body region can then be unwrapped, which is the process of unfolding an overlaid 3D mesh into a 2D texture which fits the 3D structure. This information can be converted to a map for application of various skin benefit agents, and the map can be used to create a topical application patch or mask incorporating these benefit agents. The user can then apply the topical application patch or mask to the skin surface for targeted application of the benefit agents to regions of the body surface having skin-improvement opportunities that can benefit by application of the benefit agents thereto.

In one embodiment, the body-surface data or the resulting map can be communicated to a manufacturing process that would manufacture a plurality of topical application patches. These patches can be packaged and provided to the user. One can recognize that this system can be operated through ecommerce systems incorporating the internet or can be done at a spa or small store or kiosk.

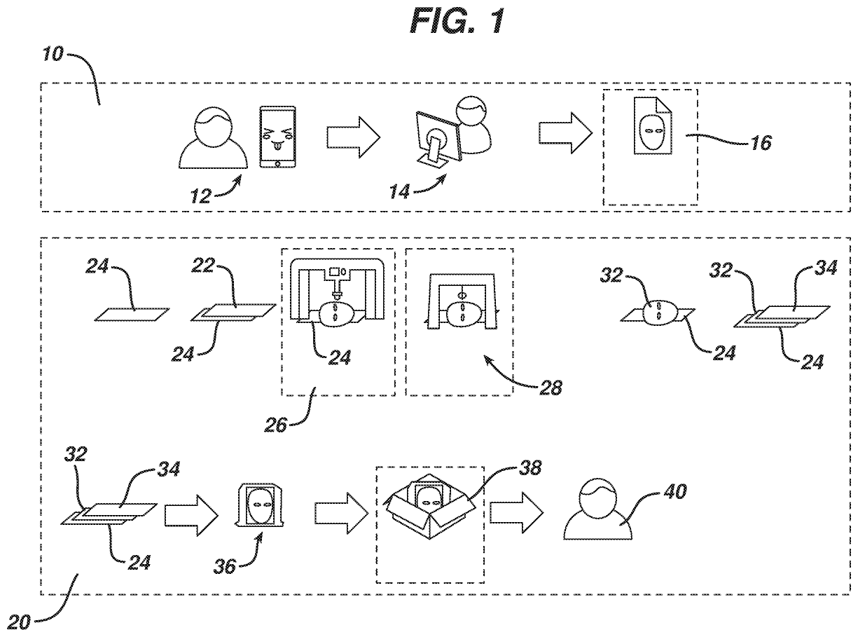

For example, as shown in FIG. 1, the design workflow 10 includes a consumer interface 12 that acquires the body-surface data which is communicated to a design creation location 14 of system to produce a digital design file 16 or map. This map 16 is communicated to a manufacturing site 20 in which a patch substrate 22 is placed on a carrier 24, one or more active benefit agents are printed (e.g., at a print station 26) on one or more regions of the patch substrate 22, the patch substrate 22 is laser cut (e.g., at a laser cutting station 28) to a desired shape for the consumer and waste material is removed. The resulting topical application patch 32 is then covered with a releasable sheet 34 and packaged for delivery to the consumer 40 (either individually in a primary package 36 or as a plurality of topical application patches in secondary packaging 38). Variations of the process will be recognized by those of ordinary skill in the art. For example, elements of the process may be performed manually—such as (1) the transformation of the body-surface data to the digital design file or map and (2) the manufacturing steps—or automatically, and the order of the steps may be varied (cutting the patch substrate may occur prior to the application of one or more active benefit agents).

In one embodiment, the carrier includes a nonwoven fabric. A representative, non-limiting list of useful nonwoven fabrics includes cellulose fabrics (derived and/or made from natural and/or regenerated fibers, such as cotton, wood pulp, rayon including viscose,): polymeric fabrics derived from renewable resources such as polylactic acid derived from corn starch, tapioca roots, sugarcane, and the like; polyolefin fabrics; polyester fabrics; and combinations thereof.

Alternatively, the carrier could be incorporated within the patch substrate, e.g., embedded within the patch substrate.

The patch substrate may therefore provide a number of functions to the personalized topical application patch. For example, it may provide an interface between a carrier material and the user's skin. It may also provide or assist adherence of the personalized topical application patch to the user's skin. Finally, it carries the active benefit agents of personalized topical application patch for delivery to the user's skin.

A preferred process to apply the active benefit agents is known as 3D printing or additive manufacturing. This permits careful control and application of active benefit agents to the patch substrate. It also permits the formation of 3D micro structures associated with the active benefit agents, such as microneedle formation to enhance penetration of the skin to deliver actives into the consumer's body.

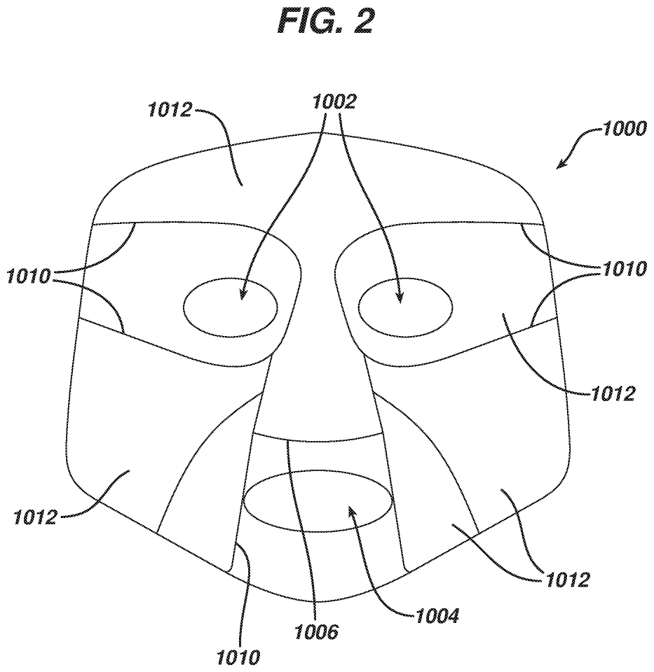

An exemplary topical application patch is a facial mask shown in FIG. 2. This shows a substantially flat mask 1000 having eye apertures 1002, a mouth aperture 1004 and a nose slit 1006. Barriers 1010 isolate regions 1012 permitting the application of active benefit agents to discrete zones of a user's face.

Many users desire to use topical application patches for facial skin improvement (also known as facial masks), but one will recognize that these personalized topical application patches can also be customized for other body surfaces, too. For example, consumers may desire using topical application patches for the chest/décolletage, hands, and other body surfaces. In addition, health practitioners may recommend or even prescribe the use of patches on other topical locations. In embodiments for the face, active skin benefit agents can be targeted for one or more of the following zones: forehead, eye orbital, nose, cheek, chin, nasolabial folds, and others.

Active benefit agents can address hydration, pigmentation and tone, redness/oxidative skin stress, wrinkles, brightening, sagging/elasticity, and acne.

A non-limiting list of useful hydrating active benefit agents includes hyaluronic acid, and humectants. The hyaluronic acid may be linear, cross-linked, or a mixture of linear and cross-linked hyaluronic acid. It may be in a salt form, such as sodium hyaluronate. The molecular weight of the hyaluronic acid may vary as desired from very low molecular weight to very high molecular weight. A commercially available cross-linked hyaluronic acid useful in the present invention is HyaCare® Filler CL from Evonik Industries AG. A humectant is a compound intended to increase the water content of the top layers of skin (e.g., hygroscopic compounds). Examples of suitable humectants include those found Chapter 35, pages 399-415 (Skin Feel Agents, by G Zocchi) in Handbook of Cosmetic Science and Technology (edited by A. Barel, M. Paye and H. Maibach, Published in 2001 by Marcel Dekker, Inc New York, N.Y.) and include, but are not limited to, glycerin, sorbitol or trehalose (e.g., alpha,alpha-trehalose, beta,beta-trehalose, alpha,beta-trehalose) or a salt or ester thereof (e.g., trehalose, 6-phosphate).

A non-limiting list of useful pigmentation active benefit agents includes resorcinols, such as niacinamide, 4-hexyl resorcinol, curcuminoids and retinoids including retinol, retinal, retinoic acid, retinyl acetate, and retinyl palmitate, enzymes such as laccase, tyrosinase inhibitors, melanin-degradation agents, melanosome transfer inhibiting agents including PAR-2 antagonists, exfoliants, sunscreens, retinoids, antioxidants, Tranexamic acid, tranexamic acid cetyl ester hydrochloride, skin bleaching agents, linoleic acid, adenosine monophosphate disodium salt, Chamomilla extract, allantoin, opacifiers, talcs and silicas, zinc salts, and the like, and other agents as described in Solano et al. Pigment Cell Res. 19 (550-571) and Ando et al. Int J Mol Sci 11 (2566-2575). Examples of suitable tyrosinase inhibitors include but, are not limited to, Vitamin C and its derivatives, Vitamin E and its derivatives, Kojic Acid, Arbutin, resorcinols, hydroquinone, Flavones e.g. Licorice flavanoids, Licorice root extract, Mulberry root extract, Dioscorea Coposita root extract, Saxifraga extract and the like, Ellagic acid, Salicylates and derivatives, Glucosamine and derivatives, Fullerene, Hinokitiol, Dioic acid, Acetyl glucosamine, 5,5′-dipropyl-biphenyl-2,2′-diol (Magnolignan), 4-(4-hydroxyphenyl)-2-butanol (4-HPB), combinations of two or more thereof, and the like. Examples of vitamin C derivatives include, but are not limited to, ascorbic acid and salts, Ascorbic Acid-2-Glucoside, sodium ascorbyl phosphate, magnesium ascorbyl phosphate, and natural extract enriched in vitamin C. Examples of vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in vitamin E derivatives. Examples of resorcinol derivatives include, but are not limited to, resorcinol, 4-substituted resorcinols like 4-alkylresorcinols such as 4-butyresorcinol (rucinol), 4-hexylresorcinol (Synovea HR, Sytheon), phenylethyl resorcinol (Symwhite, Symrise), 1-(2,4-dihydroxyphenyl)-3-(2,4-dimethoxy-3-methylphenyl)-Propane (nivitol, Unigen) and the like and natural extracts enriched in resorcinols. Examples of salicylates include, but are not limited to, 4-methoxy potassium salicylate, salicylic acid, acetylsalicylic acid, 4-methoxysalicylic acid and their salts. In certain preferred embodiments, the tyrosinase inhibitors include a 4-substituted resorcinol, a vitamin C derivative, or a vitamin E derivative

A non-limiting list of useful redness/antioxidant active benefit agents includes water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, propolis and extracts of feverfew. By “extracts of feverfew,” it is meant extracts of the plant “Tanacetum parthenium,” such as may be produced according to the details set for the in U.S. Patent Application Publication No. 2007/0196523, entitled “PARTHENOLIDE FREE BIOACTIVE INGREDIENTS FROM FEVERFEW (TANACETUM PARTHENIUM) AND PROCESSES FOR THEIR PRODUCTION.” One particularly suitable feverfew extract is commercially available as about 20% active feverfew, from Integrated Botanical Technologies of Ossining, N.Y.

A non-limiting list of useful wrinkle active benefit agents includes N-acetyl glucosamine, 2-dimethylaminoethanol, copper salts such as copper chloride, peptides like argireline, syn-ake and those containing copper, coenzyme Q10, dill, blackberry, princess tree, picia anomala, and chicory, resorcinols, such as 4-hexyl resorcinol, curcuminoids and retinoids including retinol, retinal, retinoic acid, retinyl acetate, and retinyl palmitate, hydroxy acids include, but are not limited, to glycolic acid, lactic acid, malic acid, salicylic acid, citric acid, and tartaric acid.

A non-limiting list of useful brightening active benefit agents includes Vitamin C and its derivatives such as Ascorbic Acid 2-Glucoside(AA2G), alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid.

A non-limiting list of useful benefit agents for sagging skin includes blackberry extracts, cotinus extracts, feverfew extracts, extracts of Phyllanthus niruri and bimetal complexes having copper and/or zinc constituents. The bimetal complex having copper and/or zinc constituents may be, for example, copper-zinc citrate, copper-zinc oxalate, copper-zinc tartarate, copper-zinc malate, copper-zinc succinate, copper-zinc malonate, copper-zinc maleate, copper-zinc aspartate, copper-zinc glutamate, copper-zinc glutarate, copper-zinc fumarate, copper-zinc glucarate, copper-zinc polyacrylic acid, copper-zinc adipate, copper-zinc pimelate, copper-zinc suberate, copper-zinc azealate, copper-zinc sebacate, copper-zinc dodecanoate, or combinations thereof.

A non-limiting list of useful benefit agents for acne includes benzoyl peroxide, retinoids including retinol, retinal, retinoic acid, retinyl acetate, and retinyl palmitate, hydroxy acids include, but are not limited, to glycolic acid, lactic acid, malic acid, salicylic acid, citric acid, and tartaric acid, and sulfur.

A non-limiting list of additional cosmetically acceptable active agent may be selected for instance from hydroxy acids, benzoyl peroxide, D-panthenol carotenoids, ceramides, polyunsaturated fatty acids, essential fatty acids, enzymes, such as laccase, enzyme inhibitors, minerals, hormones, such as estrogens, steroids, such as hydrocortisone, amino acids, such as proline, vitamins, lactobionic acid, acetyl-coenzyme A, niacin, riboflavin, thiamin, ribose, electron transporters, such as NADH and FADH2, natural extracts, such as those from aloe vera, feverfew, oatmeal, dill, blackberry, princess tree, picia anomala, and chicory, vitamins including but are not limited to, vitamin A, vitamin B's, such as vitamin B3, vitamin B5, and vitamin B12, vitamin C, vitamin K, and different forms of vitamin E, like alpha, beta, gamma, or delta tocopherols, or their mixtures, and derivatives thereof.

Additional skin benefit agents or actives may include those actives listed in the following paragraphs. While some of these actives may have been listed above, they are included below to ensure a more robust listing.

Examples of suitable additional active agents include: skin lightening agents, darkening agents, anti-aging agents, tropoelastin promoters, collagen promoters, anti-acne agents, shine control agents, anti-microbial agents such as anti-yeast agents, anti-fungal, and anti-bacterial agents, anti-inflammatory agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, hydration boosters, efficacy boosters, anti-callous agents, agents for skin conditioning, anti-cellulite agents, fluorides, teeth whitening agents, anti-plaque agents, and plaque-dissolving agents, odor-control agents such as odor masking or pH-changing agents, and the like. Examples of various suitable additional cosmetically acceptable actives include UV filters such as but not limited to avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, carotenoids, free radical scavengers, spin traps, retinoids and retinoid precursors such as retinol, retinoic acid and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, amino acids such a proline, vitamins, lactobionic acid, acetyl-coenzyme A, niacin, riboflavin, thiamin, ribose, electron transporters such as NADH and FADH2, and other botanical extracts such as oat, aloe vera, Feverfew, Soy, Shiitake mushroom extracts, and derivatives and mixtures thereof.

Examples of suitable skin lightening active agents include, but are not limited to, tyrosinase inhibitors, melanin-degradation agents, melanosome transfer inhibiting agents including PAR-2 antagonists, exfoliants, sunscreens, retinoids, antioxidants, Tranexamic acid, tranexamic acid cetyl ester hydrochloride, skin bleaching agents, linoleic acid, adenosine monophosphate disodium salt, Chamomilla extract, allantoin, opacifiers, talcs and silicas, zinc salts, and the like, and other agents as described in Solano et al. Pigment Cell Res. 19 (550-571) and Ando et al. Int J Mol Sci 11 (2566-2575).

Examples of suitable tyrosinase inhibitors include but, are not limited to, Vitamin C and its derivatives, Vitamin E and its derivatives, Kojic Acid, Arbutin, resorcinols, hydroquinone, Flavones e.g. Licorice flavanoids, Licorice root extract, Mulberry root extract, Dioscorea Coposita root extract, Saxifraga extract and the like, Ellagic acid, Salicylates and derivatives, Glucosamine and derivatives, Fullerene, Hinokitiol, Dioic acid, Acetyl glucosamine, 5,5′-dipropyl-biphenyl-2,2′-diol (Magnolignan), 4-(4-hydroxyphenyl)-2-butanol (4-HPB), combinations of two or more thereof, and the like. Examples of vitamin C derivatives include, but are not limited to, ascorbic acid and salts, Ascorbic Acid-2-Glucoside, sodium ascorbyl phosphate, magnesium ascorbyl phosphate, and natural extract enriched in vitamin C. Examples of vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in vitamin E derivatives. Examples of resorcinol derivatives include, but are not limited to, resorcinol, 4-substituted resorcinols like 4-alkylresorcinols such as 4-butyresorcinol (rucinol), 4-hexylresorcinol (Synovea HR, Sytheon), phenylethyl resorcinol (Symwhite, Symrise), 1-(2,4-dihydroxyphenyl)-3-(2,4-dimethoxy-3-methylphenyl)-Propane (nivitol, Unigen) and the like and natural extracts enriched in resorcinols. Examples of salicylates include, but are not limited to, 4-methoxy potassium salicylate, salicylic acid, acetylsalicylic acid, 4-methoxysalicylic acid and their salts. In certain preferred embodiments, the tyrosinase inhibitors include a 4-substituted resorcinol, a vitamin C derivative, or a vitamin E derivative. In more preferred embodiments, the tyrosinase inhibitor comprises Phenylethyl resorcinol, 4-hexyl resorcinol, or ascorbyl-2-glucoside.

Examples of suitable melanin-degradation agents include, but are not limited to, peroxides and enzymes such as peroxidases and ligninases. In certain preferred embodiments, the melanin-inhibiting agents include a peroxide or a ligninase.

Examples of suitable melanosome transfer inhibiting agents including PAR-2 antagonists such as soy trypsin inhibitor or Bowman-Birk Inhibitor, Vitamin B3 and derivatives such as Niacinamide, Essential soy, Whole Soy, Soy extract. In certain preferred embodiments, the melanosome transfer inhibiting agents includes a soy extract or niacinamide.

Examples of exfoliants include, but are not limited to, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid, and mechanical exfoliation such as microdermabrasion. In certain preferred embodiments, the exfoliant include glycolic acid or salicylic acid.

Examples of sunscreens include, but are not limited to, avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, and the like.

Examples of retinoids include, but are not limited to, retinol (Vitamin A alcohol), retinal (Vitamin A aldehyde), retinyl acetate, retinyl propionate, retinyl linoleate, retinoic acid, retinyl palmitate, isotretinoin, tazarotene, bexarotene, Adapalene, combinations of two or more thereof and the like. In certain preferred embodiments, the retinoid is selected from the group consisting of retinol, retinal, retinyl acetate, retinyl propionate, retinyl linoleate, and combinations of two or more thereof. In certain more preferred embodiments, the retinoid is retinol.

Examples of antioxidants include, but are not limited to, water-soluble antioxidants such as sulfhydryl compounds and their derivatives (e.g., sodium metabisulfite and N-acetyl-cysteine, glutathione), lipoic acid and dihydrolipoic acid, stilbenoids such as resveratrol and derivatives, lactoferrin, iron and copper chelators and ascorbic acid and ascorbic acid derivatives (e.g., ascobyl-2-glucoside, ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinones. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, black tea, white tea, pine bark, feverfew, parthenolide-free feverfew, oat extracts, blackberry extract, cotinus extract, soy extract, pomelo extract, wheat germ extract, Hesperedin, Grape extract, Portulaca extract, Licochalcone, chalcone, 2,2′-dihydroxy chalcone, Primula extract, propolis, and the like.

In addition to the foregoing exemplary active benefit agents, above, persons of ordinary skill will recognize that other components may be incorporated into the personalized topical application patch, including without limitation, additional film-formers, plasticizers, pigments and opacifiers, preservatives, fragrances, and other components desired by a formulator.

A personalized (alternatively customized) topical application patch useful in the above system may be manufactured while immobilizing one or more actives associated with a patch substrate, such as a hydrogel, to facilitate spatial segregation of the actives. Such immobilization can either be completed through covalent attachment or immiscibility characteristics (i.e., placement of hydrophobic active). While these strategies will facilitate spatial segregation, the diffusion out of the hydrogel will be limited and reduce the efficacy of any associated treatment. We have identified improved spatial control of actives, including water soluble actives, within a substrate, such as a hydrogel substrate, can be controlled through a fabrication process and creation of barriers between areas of customization without reducing the diffusion of actives to a user's skin during use.

For oil soluble, partially water soluble, or water insoluble active benefit agents a microemulsion with an external hydrophilic phase can be used as the formulation. The resulting microemulsion with an external hydrophilic phase containing oil soluble, partially water soluble, or water insoluble active benefit agents can be printed on the regions of mask wherein the formulation contains one or more of the benefit agents.

The active benefit agents can be incorporated into the personalized topical application patch by methods known to those of ordinary skill in the art including without limitation, printing, spraying, coating, and the like. Water soluble active benefit agent compositions are readily incorporated into a hydrogel patch substrate due to their hydrophilic character. Oil soluble, partially water soluble, or water insoluble active benefit agents can be incorporated into an emulsion or a microemulsion with an external hydrophilic phase can be used to incorporate these less soluble active benefit agent compositions.

As indicated above, active benefit agents may be sprayed as powder, liquid or suspension onto the surface of the patch substrate. Such spray applications may result in a coating of the surface of the patch substrate which could concentrate the benefit agents at the surface of the patch surface. Alternatively, with greater hydrophilicity and/or aqueous carrier, the sprayed composition may also migrate deeper into a hydrogel patch substrate.

Utilizing a barrier approach between areas of customization, the diffusion of actives within the hydrogel can be minimized. This may be accomplished through several general strategies described below.

In one embodiment, the diffusion of actives may be minimized through viscosity modification. Compounds that can be used to increase the effective viscosity of the matrix may limit diffusion of the water soluble active through diffusion control. This may be accomplished through mechanisms consistent with gelatin (or gelatinous compounds) whereby viscosity control can be accomplished through temperature modulation. A construct for the active can be achieved through placement of the active within the gelatin matrix (either as applied or a 2-step process). The active will remain in position until application of the product. The phase change occurring during application from temperature (either through body temperature or external application) would reduce the viscosity and allow the active to diffusion from the hydrogel.

In another embodiment, the of forming the barriers is achieved by depositing physically discrete benefit agent-containing matrices onto the patch substrate. The benefit agent-containing matrices may be high viscosity matrix materials such as gelatin.

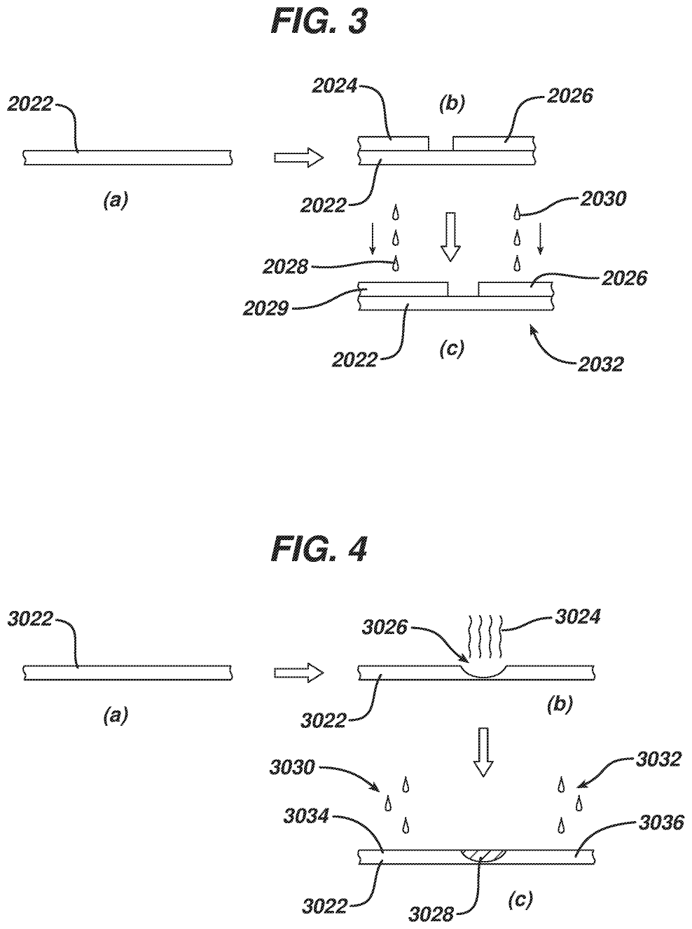

An image of potential application strategies is shown in FIG. 3. As shown in step (a), a hydrogel patch substrate 2022 is provided. In step (b) gelatin layers can be applied to desired treatment zones 2024, 2026, and subsequently, active agents 2028, 2030 are applied (step (c)). Subsequently, the resulting topical application patch 2032 can then be applied to the skin of the user. In an alternative embodiment in which the active agents 2028, 2030 are not temperature sensitive, the active agents 2028 and 2030 can be included in the gelatin layers 2024, 2026 (combining steps (b) and (c)). The concept described through the mechanism has the additional benefit of temperature targeted release. As the gelatin-like construct can be designed such that the phase transition between gel and liquid is targeted for a specific treatment. These treatments may include skin activation either through normal body temperature or fever condition, specific areas within the GI tract, thermally activated from an external source (either internal or external to the body), and oral activation (i.e., through warm fluid application). Specific compounds that may be used include xanthum, agar, chitosan, carrageenan, etc.

Additional mechanism of phase transition may include the counterion exchange and/or pH change. In the construct where the hydrogel is formed through divalent counterions, substitution of a monovalent counterion will cause a change in crosslinking and/or viscosity allowing the transfer of active. Mechanism may include activation through application of NaCl solution or similar components to a construct crosslinked via Mg+2, Ca+2 or similar system. In a similar fashion, albeit through disruption of hydrogen bonding and solubility, pH change may be used as a stimulus for active release. Hydrogels formed through hydrogen bonding may be displaced through pH change effectively lowering the viscosity for release. Additionally, inclusion of long chain fatty acids as viscosity modifiers (e.g., hexanoic acid, decanoic acid, etc.) may facilitate a lower viscosity through solubility change with an increase in pH. The increased solubility will allow molecular mobility within the hydrogel and release of the active.

In addition to the x-y segregation detailed in the image above, the concept can be applied to a multi-layer construct for pulsatile or controlled release. The phase transition occurring upon the application of stimulus (e.g., heat, counterion exchange, etc.) may lead to the effective dissolution of a single layer releasing the active. The subsequent layer in the z axis may be a constructed of a layer requiring an alternative stimulus than the first. This could be repeated through the construct until complete dissolution of the active layers.

In another embodiment, the diffusion of actives may be minimized through creation of hydrophobic barriers. Mitigation of diffusion for a water-soluble component spatially may be accomplished through the effective dehydration of the hydrogel along a barrier and application of hydrophobic species to minimize rehydration. This can be accomplished via selective dehydration from target thermal application (e.g., low power laser etching, directed IR heat, directed microwave radiation, etc.) that forms the dehydrated zone. Subsequently, a hydrophobic component (e.g., silicone oil, etc.) can be added to the dehydrated area to form the barrier. Upon rehydration, zones of segregated hydrogel can be formed. A schematic of this process is shown in FIG. 4.

As shown in step (a), a hydrogel patch substrate 3022 is provided. In step (b) heat 3024 is applied to dehydrate a portion of the hydrogel patch substrate 3026. In step (c), a hydrophobic component 3028 and active agents 3030, 3032 are applied to desired treatment zones 3034, 3036 are applied to the hydrogel patch substrate 3022.

This can be accomplished through dehydration of the entire hydrogel as well such that the hydrophobic species is added before hydration.

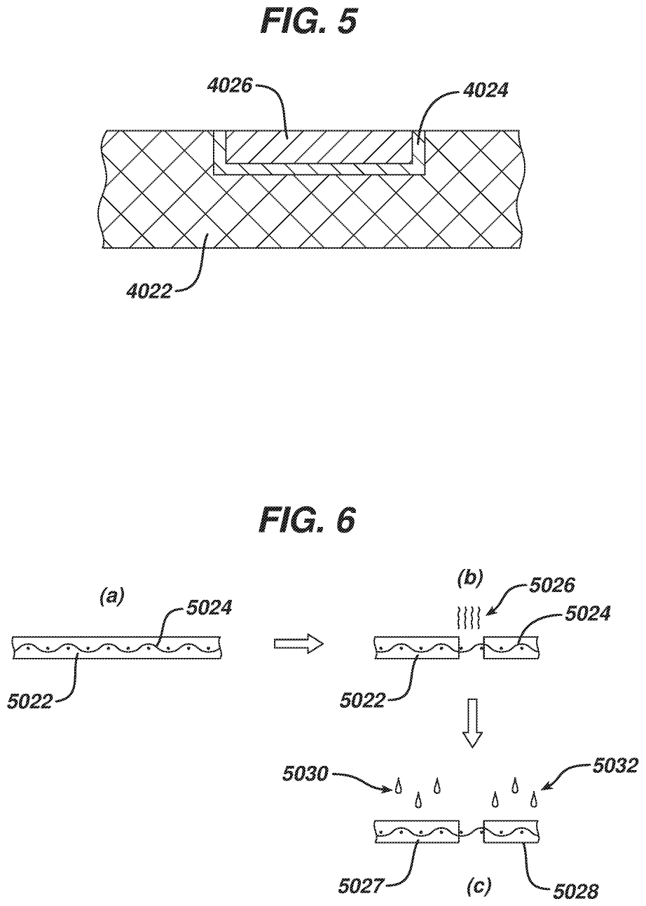

In addition to the inclusion of a gross hydrophobic barrier, a thin film hydrophobic barrier may be utilized. A schematic of the construct is shown in FIG. 5 in which the hydrogel substrate 4022 has includes a hydrophobic barrier 4024 to isolate a portion with a hydrogel with active agent 4026.

The utilization of the hydrophobic barrier prevents the segregation of the active through potential diffusions below the selected hydration layer. As there is a potential for the hydrogel with the active to delaminate from the bulk hydrogel (i.e., limited adhesion through the hydrophobic layer), there is a need to adhere and/or covalently attach the hydrophobic barrier to both hydrogels. This may be completed through hydrogel surface modification via surface catalyzed polymerization whereby a hydrophobic chain end is added to the surface via ring opening polymerization, etc. The resultant chain end can be functionalized to covalently attached to the hydrogel with active. The formed triblock copolymer will contain a hydrophobic center unit that will serve as the barrier. Similar technology could be used via incorporation of the triblock as an additional process step between the selective dehydration and application of the hydrogel with active.

In an alternative embodiment, the diffusion of actives may be minimized through physically separating actives. For hydrogels with imbedded meshes or reinforcement agents, spatially segregation of water soluble components can be achieved through eliminating the continuity of the hydrogel. This can be done through similar technologies as those described for the selective dehydration at higher temperatures such that the hydrogel is ablated either through degradation or elimination of hydrogen bonding (i.e., eliminate gel formation such that the viscous fluid can be removed). The mesh or reinforcement should maintain the integrity of the product while allowing segregation of the active component areas. A schematic of the process is shown in FIG. 6.

As shown in step (a), a hydrogel patch substrate 5022 incorporates a fabric mesh 5024. In step (b) heat 5026 is applied to remove a portion of the hydrogel patch substrate, leaving the fabric mesh 5024 to maintain the relative location of treatment zones 5027, 5028. In step (c), active agents 5030, 5032 are applied to desired treatment zones 5027, 5028 are applied to the hydrogel patch substrate 5022. The gap between patch treatment zones 5027, 5028 acts as the barrier between the hydrogel zones.

In another embodiment, the diffusion of actives may be minimized through selective crosslinking of crosslinkable materials. For hydrogels with labile hydrogens, acrylic functionality, dissociated hydrogel bonding, etc., an increase in hydrogel crosslinking may be possible. The increase in crosslinking may reduce the water content, entrap the active, and/or increase molecular density. Selectively crosslinking patch substrate material can be leveraged to form barriers within the hydrogel to prevent diffusion of the active. The form of the crosslinking can be covalent (i.e., chemical reaction to form bond between atoms), counterion (e.g., utilization of divalent ions to access intermolecular forces, etc.), induced by a radiation source, including without limitation electron beam, UV, gamma, and the like, and/or hydrogen bonding.

The present invention will be further understood by reference to the following specific Examples which are illustrative of the composition, form and method of producing the present invention. It is to be understood that many variations of composition, form and method of producing this would be apparent to those skilled in the art. The following Examples, wherein parts and percentages are by weight unless otherwise indicated, are only illustrative.

### EXAMPLES

### Example 1: Hydrogel Composition

An example of a hydrogel preparation according to the invention used the ingredients shown in Table 1.

TABLE 1
Composition 1

| | | Percentage (wt-%, |
|---|---|---|
| | | rounded to nearest |
| INCI Name | Trade Name | 0.1%) |
| WATER | PURIFIED | 89.3 |
| | WATER | |
| Carrageenan | Genugel CG-130 | 1.2 |
| Ceratonia Siliqua | Cesagum LN-1 | 0.6 |
| (Carob) Gum | | |
| Glycerin | Moon OU Kosher | 7.5 |
| | Glycerin, | |
| | USP/FCC | |
| Potassium Sorbate | Potassium | 0.6 |
| | Sorbate Granular, | |
| | EMPROVE, Ph | |
| | Eur, BP, NF, FCC | |
| | E202 | |
| Chlorphenesin | Cosvat | 0.3 |
| Phenoxyethanol | Phenoxyethanol | 0.3 |
| | P25/Tristat P25 | |
| Ethylhexylglycerin | Sensivia SC-50 | 0.3 |

Composition 1 was prepared as follows.

Glycerin Pre-Mix
Step 1. Glycerin, Carrageenan and Ceratonia Siliqua (Carob) Gum were placed into a beaker. The composition was stirred with a spatula until uniform consistency was reached.

Water Phase
Step 1. In a second beaker, Water was heated to 85° C.
Step 2. Potassium sorbate, Chlorphenesin, Phenoxyethanol, and Ethylhexylglycerin were added to the Water in the second beaker and mixed until all components are well dissolved/dispersed.

Full Mixture
Step 1. The aqueous mixture (“Water Phase,” above) was added to the Glycerin Pre-Mix, while stirring and maintaining composition solution at 85° C. for a minimum of 10 minutes, to form a hydrogel having a uniform consistency.
Step 2. The temperature was maintained at 85° C. until the hydrogel was cast (in Step 3, below).
Step 3. To cast, a desired amount of hydrogel was poured onto a pre-heated surface or mold with the desired shape. The hydrogel was cooled to room temperature (˜25 C), and the mold was removed.

### Examples 2 & 3: Active Printable Compositions

The application of the active benefit agents to the patch substrate can be achieved by printed on one or more regions of the patch substrate. For water soluble active benefit agents a formulation can be printed on the regions of mask wherein the formulation contains one or more of the benefit agents. An example of a printable formulation containing a water-soluble benefit agent is shown below:

The following compositions according to the invention, Compositions 2&3, were prepared using the ingredients shown in Table 2&3, respectively.

TABLE 2
Composition 2

| | | Percentage (wt-%, |
|---|---|---|
| | | rounded to nearest |
| INCI Name | Trade Name | 0.1%) |
| WATER | PURIFIED | 65.8 |
| | WATER | |
| Niacinamide | Niacinamide PC | 33.3 |
| Chlorphenesin | Cosvat | 0.3 |
| Phenoxyethanol | Phenoxyethanol | 0.3 |
| | P25/Tristat P25 | |
| Ethylhexylglycerin | Sensivia SC-50 | 0.3 |

Niacinamide, Chlorphenesin, Phenoxyethanol and Ethylhexylglycerin were dispersed in water, and mixed until all ingredients fully dissolved (some slight heating assisted in the dispersion).

TABLE 3
Composition 3

| INCI Name | Trade Name | Percentage |
|---|---|---|
| WATER | PURIFIED | 28.1 |
| | WATER | |
| Glycerin | Moon OU Kosher | 28.1 |
| | Glycerin, | |
| | USP/FCC | |
| N-Acetyl D-Glucosamine | Acetyl | 42.9 |
| | Glucosamine | |
| Chlorphenesin | Cosvat | 0.3 |
| Phenoxyethanol | Phenoxyethanol | 0.3 |
| | P25/Tristat P25 | |
| Ethylhexylglycerin | Sensivia SC-50 | 0.3 |

To prepare composition, add Acetyl Glucosamine to a beaker. Glycerin, Water, Chlorphenesin, Phenoxyethanol and Ethylhexylglycerin were added to a beaker already containing the Acetyl Glucosamine. The composition was stirred with a magnetic stir bar until all components were well dispersed.

What is claimed is:

1. A method for providing a personalized topical application patch to a person comprising the steps of:
a) acquiring body-surface data of a person;
b) communicating the body-surface data to a design creation system which produces a digital design file corresponding to the body-surface data;
c) communicating the digital design file to a manufacturing site;
d) forming a personalized topical application patch by:
i) placing a patch substrate on a carrier;
ii) forming at least one barrier in the patch substrate to define at least two discrete regions of the patch substrate;
iii) applying one or more active benefit agents onto at least one of the discrete regions of the patch substrate; and
iv) cutting the patch substrate to a desired shape for the person and removing waste material patch substrate material;
wherein the barriers are substantially impervious to diffusion of the one or more active benefit agents;
e) packaging the personalized topical application patch; and
f) delivering the personalized topical application patch to the person.

2. The method of claim 1 wherein the barriers are hydrophobic.

3. The method of claim 1 wherein the patch substrate comprises a hydrogel and the step of forming the barriers comprises dehydrating the hydrogel.

4. The method of claim 1 wherein the patch substrate comprises a hydrogel and the step of forming the barriers comprises eliminating the continuity of the hydrogel.

5. The method of claim 1 wherein the patch substrate comprises a crosslinkable material and the step of forming the barriers comprises selectively crosslinking patch substrate material.

6. The method of claim 1 wherein the step of forming the barriers comprises depositing physically discrete benefit agent-containing matrices onto the patch substrate.

7. The method of claim 6 wherein the benefit agent-containing matrices comprise a high viscosity matrix material.

8. The method of claim 7 wherein the high viscosity matrix material comprises gelatin.

9. A personalized topical application patch comprising:
a) a patch substrate having a plurality of isolated regions;
b) one or more active benefit agents disposed at least one of the plurality of isolated regions; and
c) at least one barrier disposed between adjacent isolated regions, wherein the at least one barrier is substantially impervious to diffusion of the one or more active benefit agents.

10. The patch of claim 9 wherein the at least one barrier is hydrophobic.

11. The patch of claim 9 wherein the patch substrate comprises a hydrogel and the at least one barrier comprises dehydrated hydrogel.

12. The patch of claim 9 wherein the patch substrate comprises a hydrogel and at least one barrier comprises a gap in the hydrogel.

13. The patch of claim 9 wherein the patch substrate comprises a crosslinkable material and the at least one barrier comprises selectively crosslinking patch substrate material.

14. The patch of claim 9 wherein the one or more active benefit agents are disposed in physically discrete benefit agent-containing matrices on the patch substrate.

15. The patch of claim 14 wherein the benefit agent-containing matrices comprise a high viscosity matrix material.

16. The patch of claim 15 wherein the high viscosity matrix material comprises gelatin.
